(19) United States
(12) Patent Application Publication
(10) Pub. No.: US 2026/0240528 A1
(43) Pub. Date: Aug. 20, 2026

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Kaito TAKASHIMA, Kanagawa (JP)

(73) Assignee: FUJIFILM Corporation, Tokyo (JP)

(21) Appl. No.: 19/430,310

(22) Filed: Dec. 23, 2025

(30) Foreign Application Priority Data
Feb. 19, 2025 (JP) 2025-024779

(51) Int. Cl. A61B 8/00; A61B 8/08

(52) U.S. Cl. CPC A61B 8/54; A61B 8/0883; A61B 8/42; A61B 8/488

(57) ABSTRACT

Provided are an ultrasound diagnostic apparatus and a control method of an ultrasound diagnostic apparatus that can dispose a Doppler gate at an appropriate position to obtain an accurate Doppler measurement value.

An ultrasound diagnostic apparatus includes a gate setting unit that sets a Doppler gate on an ultrasound image, a boundary recognition unit that recognizes a boundary between a ventricular septum and a left ventricular cavity in the ultrasound image, an overlap determination unit that determines whether or not an overlap between a scanning line passing through a center point of the Doppler gate and the recognized boundary is present, a time-series change determination unit that determines whether or not an overlap determination result changes within one cardiac cycle, and an avoidance guidance unit that guides a user in a method for avoiding a change in the overlap determination result in a case where it is determined that the overlap determination result changes within the one cardiac cycle.

### CROSS-REFERENCE TO RELATED APPLICATIONS

The present application claims priority under 35 U.S.C. § 119 to Japanese Patent Application No. 2025-024779, filed on Feb. 19, 2025. The above application is hereby expressly incorporated by reference, in its entirety, into the present application.

### BACKGROUND OF THE INVENTION

### 1. Field of the Invention

The present invention relates to an ultrasound diagnostic apparatus that images a heart of a subject and a control method of the ultrasound diagnostic apparatus. 2. Description of the Related Art

In the related art, a so-called cardiac output is calculated by imaging an ultrasound image representing a tomographic plane of a heart of a subject using a so-called ultrasound diagnostic apparatus and analyzing the imaged ultrasound image. The cardiac output is usually calculated by performing calculation steps of (1) measuring a diameter of a left ventricular outflow tract in an ultrasound image representing a so-called parasternal left ventricular long-axis cross section at a mid-systolic phase of the heart, and calculating a cross-sectional area of the left ventricular outflow tract, (2) calculating a velocity time integral value of a blood flow of the left ventricular outflow tract with respect to a so-called apical five-chamber cross section by a so-called pulse Doppler method, (3) calculating a so-called stroke volume by multiplying the cross-sectional area of the left ventricular outflow tract by the velocity time integral value of the blood flow of the left ventricular outflow tract, and (4) calculating the cardiac output by multiplying the stroke volume by a heart rate.

In a step of calculating the cardiac output, in a case of calculating the velocity time integral value of the blood flow of the left ventricular outflow tract by the pulse Doppler method, it is necessary to dispose a so-called Doppler gate on the left ventricular outflow tract in the ultrasound image representing the apical five-chamber cross section. A user having a low level of skill in the examination may have difficulty disposing the Doppler gate at an appropriate position on the ultrasound image. Therefore, for example, a technique disclosed in JP6987048B has been developed. JP6987048B discloses that a region of interest is automatically set for the left ventricular outflow tract in the ultrasound image, a plurality of candidates of the Doppler gate are set in the set region of interest, and an optimal position of the Doppler gate is selected based on a Doppler spectrum waveform calculated for each of the plurality of candidates of the Doppler gate.

### SUMMARY OF THE INVENTION

Meanwhile, the Doppler measurement value obtained in the Doppler gate is affected by a structure overlapping a scanning line passing through a center point of the Doppler gate. For example, in a case where there is an obstacle such as a so-called ventricular septum or a calcified portion on the scanning line, or the like, in a case where a state in which the scanning line passing through a boundary between the ventricular septum and a left ventricular cavity and the center point of the Doppler gate overlap each other and a state in which the scanning line passing through the boundary and the center point of the Doppler gate are separated from each other alternately appear in time series due to pulsation of the heart of the subject, an accurate Doppler measurement value may not be obtained. Even in a case where the position of the Doppler gate is selected by using the technique of JP6987048B, there is a case where an accurate Doppler measurement value is not obtained due to the overlap of the scanning line passing through the center point of the Doppler gate with the obstacle.

The present invention has been made to solve such a problem in the related art, and an object of the present invention is to provide an ultrasound diagnostic apparatus and a control method of an ultrasound diagnostic apparatus that can dispose a Doppler gate at an appropriate position to obtain an accurate Doppler measurement value.

The above object can be achieved with the following configurations.
[1] An ultrasound diagnostic apparatus comprising:
an ultrasound probe;
an image acquisition unit that acquires an ultrasound image in which a heart of a subject is imaged by transmitting and receiving an ultrasound beam using the ultrasound probe;
a gate setting unit that sets a Doppler gate on the ultrasound image;
a boundary recognition unit that recognizes a boundary between a ventricular septum and a left ventricular cavity in the ultrasound image;
an overlap determination unit that determines whether or not an overlap between a scanning line passing through a center point of the Doppler gate set by the gate setting unit and the boundary recognized by the boundary recognition unit is present;
a time-series change determination unit that determines whether or not an overlap determination result of the overlap determination unit changes within one cardiac cycle; and an avoidance guidance unit that guides a user in a method for avoiding a change in the overlap determination result in a case where the time-series change determination unit determines that the overlap determination result changes within the one cardiac cycle.
[2] The ultrasound diagnostic apparatus according to [1], in which the avoidance guidance unit guides movement of a position of the Doppler gate on the ultrasound image.
[3] The ultrasound diagnostic apparatus according to [2], in which the avoidance guidance unit includes an update position calculation unit that calculates an update position of the Doppler gate at which the scanning line and the boundary do not overlap each other over the one cardiac cycle.
[4] The ultrasound diagnostic apparatus according to [3], in which the avoidance guidance unit guides the user to the update position calculated by the update position calculation unit.
[5] The ultrasound diagnostic apparatus according to [3], in which the avoidance guidance unit causes the gate setting unit to set a new Doppler gate based on the update position calculated by the update position calculation unit.
[6] The ultrasound diagnostic apparatus according to [1], in which the avoidance guidance unit guides movement of the ultrasound probe.
[7] The ultrasound diagnostic apparatus according to [6], in which the avoidance guidance unit guides the ultrasound probe to slide in a right flank direction of the subject.
[8] The ultrasound diagnostic apparatus according to [1], in which the image acquisition unit acquires the ultrasound image in which an apical five-chamber cross section is imaged, and
the avoidance guidance unit guides measurement in an apical three-chamber cross section.
[9] A control method of an ultrasound diagnostic apparatus comprising:
acquiring an ultrasound image in which a heart of a subject is imaged by transmitting and receiving an ultrasound beam using an ultrasound probe;
setting a Doppler gate on the ultrasound image;
recognizing a boundary between a ventricular septum and a left ventricular cavity in the ultrasound image;
determining whether or not an overlap between a scanning line passing through a center point of the set Doppler gate and the recognized boundary is present;
determining whether a determination result regarding the presence or absence of overlap changes within one cardiac cycle; and
guiding a user in a method for avoiding a change in the determination result regarding the presence or absence of overlap in a case where it is determined that the determination result regarding the presence or absence of overlap changes within the one cardiac cycle.

In the present invention, since the ultrasound diagnostic apparatus includes an ultrasound probe, an image acquisition unit that acquires an ultrasound image in which a heart of a subject is imaged by transmitting and receiving an ultrasound beam using the ultrasound probe, a gate setting unit that sets a Doppler gate on the ultrasound image, a boundary recognition unit that recognizes a boundary between a ventricular septum and a left ventricular cavity in the ultrasound image, an overlap determination unit that determines whether or not an overlap between a scanning line passing through a center point of the Doppler gate set by the gate setting unit and the boundary recognized by the boundary recognition unit is present, a time-series change determination unit that determines whether or not an overlap determination result of the overlap determination unit changes within one cardiac cycle, and
an avoidance guidance unit that guides a user in a method for avoiding a change in the overlap determination result in a case where the time-series change determination unit determines that the overlap determination result changes within the one cardiac cycle, the Doppler gate can be disposed at an appropriate position to obtain an accurate Doppler measurement value.

### BRIEF DESCRIPTION OF THE DRAWINGS

FIG. 1 is a block diagram showing a configuration of an ultrasound diagnostic apparatus according to an embodiment of the present invention.

FIG. 2 is a block diagram showing an internal configuration of a transmission/reception circuit in the embodiment of the present invention.

FIG. 3 is a block diagram showing an internal configuration of an image generation unit in the embodiment of the present invention.

FIG. 4 is a diagram showing an example of an ultrasound image representing an apical five-chamber cross section.

FIG. 5 is a diagram showing a boundary between a ventricular septum and a left ventricular cavity in the ultrasound image representing the apical five-chamber cross section.

FIG. 6 is a diagram showing an example of a scanning line overlapping the boundary between the ventricular septum and the left ventricular cavity in the ultrasound image representing the apical five-chamber cross section.

FIG. 7 is a block diagram showing an internal configuration of an avoidance guidance unit in the embodiment of the present invention.

FIG. 8 is a diagram showing an example of guidance by the avoidance guidance unit.

FIG. 9 is a block diagram showing an internal configuration of a velocity time integral value calculation unit in the embodiment of the present invention.

FIG. 10 is a flowchart showing an operation of the ultrasound diagnostic apparatus according to an embodiment of the present invention.

### DESCRIPTION OF THE PREFERRED EMBODIMENTS

Hereinafter, embodiments of the present invention will be described based on the accompanying drawings.

The following configuration requirements are described based on a representative embodiment of the present invention, but the present invention is not limited to the embodiment.

In the present specification, a numerical range represented by “to” means a range including numerical values described before and after “to”, both ends inclusive, as a lower limit value and an upper limit value.

In the present specification, the terms “same” and “identical” include an error range that is generally allowed in the technical field.

### Embodiment

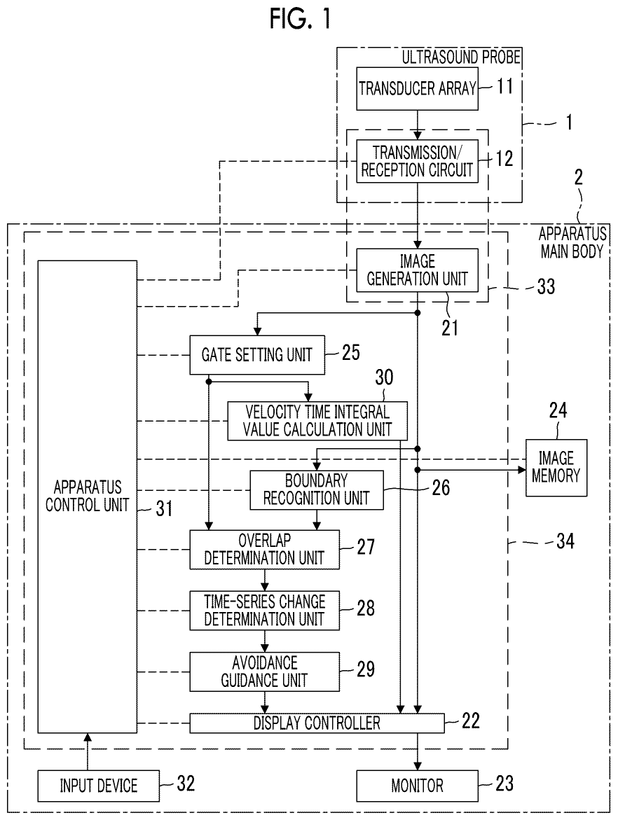

FIG. 1 shows a configuration of an ultrasound diagnostic apparatus according to an embodiment of the present invention. The ultrasound diagnostic apparatus comprises an ultrasound probe 1 and an apparatus main body 2 that are connected to each other by so-called wired communication or so-called wireless communication.

The ultrasound probe 1 comprises a transducer array 11 and a transmission/reception circuit 12 connected to the transducer array 11.

The apparatus main body 2 comprises an image generation unit 21 connected to the transmission/reception circuit 12. In the apparatus main body 2, a display controller 22 and a monitor 23 are sequentially connected to the image generation unit 21. An image memory 24, a gate setting unit 25, and a boundary recognition unit 26 are connected to the image generation unit 21. An overlap determination unit 27 is connected to the gate setting unit 25 and the boundary recognition unit 26. A time-series change determination unit 28 and an avoidance guidance unit 29 are sequentially connected to the overlap determination unit 27. The avoidance guidance unit 29 is connected to the display controller 22. In addition, a velocity time integral value calculation unit 30 is connected to the gate setting unit 25. The velocity time integral value calculation unit 30 is connected to the display controller 22. In addition, an apparatus control unit 31 is connected to the transmission/reception circuit 12, the image generation unit 21, the display controller 22, the image memory 24, the gate setting unit 25, the boundary recognition unit 26, the overlap determination unit 27, the time-series change determination unit 28, the avoidance guidance unit 29, and the velocity time integral value calculation unit 30. An input device 32 is connected to the apparatus control unit 31.

The transmission/reception circuit 12 and the image generation unit 21 constitute an image acquisition unit 33. In addition, a processor 34 for the apparatus main body 2 is configured by the image generation unit 21, the display controller 22, the gate setting unit 25, the boundary recognition unit 26, the overlap determination unit 27, the time-series change determination unit 28, the avoidance guidance unit 29, the velocity time integral value calculation unit 30, and the apparatus control unit 31.

The transducer array 11 of the ultrasound probe 1 has a plurality of ultrasonic transducers arranged in a one-dimensional or two-dimensional manner. In accordance with a drive signal supplied from the transmission/reception circuit 12, each of the ultrasound transducers transmits ultrasound and receives an ultrasound echo from a subject to output a signal based on the ultrasound echo. Each ultrasound transducer is configured by, for example, forming electrodes at both ends of a piezoelectric material consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), and the like.

The image acquisition unit 33, which is composed of the transmission/reception circuit 12 and the image generation unit 21, acquires ultrasound images of a plurality of frames as a moving image in which a heart of the subject is imaged, by transmitting and receiving ultrasound beams using the ultrasound probe 1.

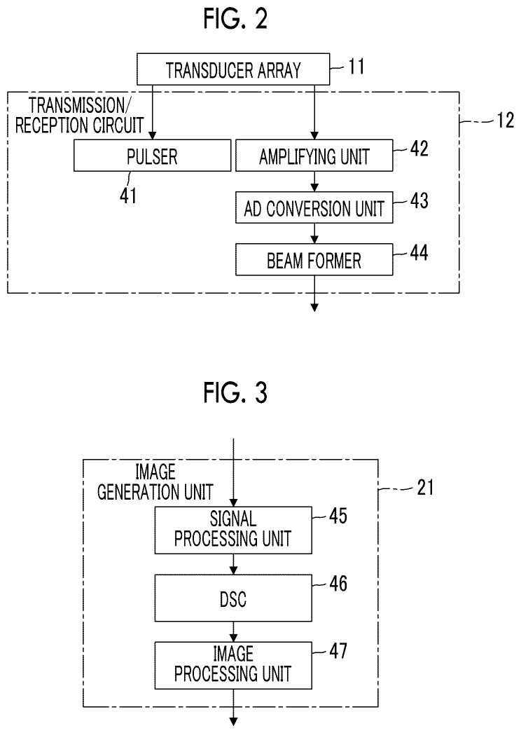

The transmission/reception circuit 12 transmits the ultrasound waves from the transducer array 11 and generates a sound ray signal based on reception signals acquired by the transducer array 11 under control of the apparatus control unit 31. As shown in FIG. 2, the transmission/reception circuit 12 includes a pulser 41 connected to the transducer array 11, and an amplifying unit 42, an analog-to-digital (AD) conversion unit 43, and a beam former 44 that are sequentially connected in series to the transducer array 11.

The pulser 41 includes, for example, a plurality of pulse generators, adjusts a delay amount of each drive signal based on a transmission delay pattern selected in accordance with a control signal from the apparatus control unit 31 so that the ultrasound waves transmitted from the plurality of ultrasound oscillators of the transducer array 11 form an ultrasound beam, and supplies each drive signal to the plurality of ultrasound oscillators. As described above, in a case in which a pulsed or continuous wave-like voltage is applied to the electrodes of the ultrasound transducer of the transducer array 11, the piezoelectric material expands and contracts to generate pulsed or continuous wave-like ultrasound from each of the ultrasound transducers, whereby the ultrasound beam is formed from the combined wave of the ultrasound.

The transmitted ultrasound beam is, for example, reflected by a target such as a part of the subject and propagates toward the transducer array 11 of the ultrasound probe 1. The ultrasound echo propagating toward the transducer array 11 in this way is received by each of the ultrasound transducers constituting the transducer array 11. In such a case, each of the ultrasound transducers constituting the transducer array 11 receives the propagating ultrasound echo to expand and contract, generates the reception signal, which is an electrical signal, and outputs these reception signals to the amplifying unit 42.

The amplifying unit 42 amplifies the signal input from each of the ultrasound transducers constituting the transducer array 11 and transmits the amplified signal to the AD conversion unit 43. The AD conversion unit 43 converts the signal transmitted from the amplifying unit 42 into digital reception data. The beam former 44 performs so-called reception focus processing by applying and adding the delay to each reception data received from the AD conversion unit 43. By the reception focus processing, each reception data, which is converted by the AD conversion unit 43, is phase-added, and the sound ray signal in which the focus of the ultrasound echo is narrowed down is acquired.

As shown in FIG. 3, the image generation unit 21 has a configuration in which a signal processing unit 45, a digital scan converter (DSC) 46, and an image processing unit 47 are sequentially connected in series.

The signal processing unit 45 corrects attenuation by distance of the sound ray signal received from the transmission/reception circuit 12 in accordance with depths of reflection positions of the ultrasound waves using a sound speed value set by the apparatus control unit 31 and then performs envelope detection processing on the sound ray signal to generate a B-mode image signal that is tomographic image information related to tissues inside the subject.

The DSC 46 converts (raster-converts) the B-mode image signal, which is generated by the signal processing unit 45, into the image signal in accordance with a normal television signal scanning method.

The image processing unit 47 performs various types of necessary image processing such as gradation processing on the B-mode image signal input from the DSC 46, and then transmits the B-mode image signal to the display controller 22, the image memory 24, the gate setting unit 25, and the boundary recognition unit 26. Hereinafter, the B-mode image signal, which is image-processed by the image processing unit 47, will be referred to as an ultrasound image.

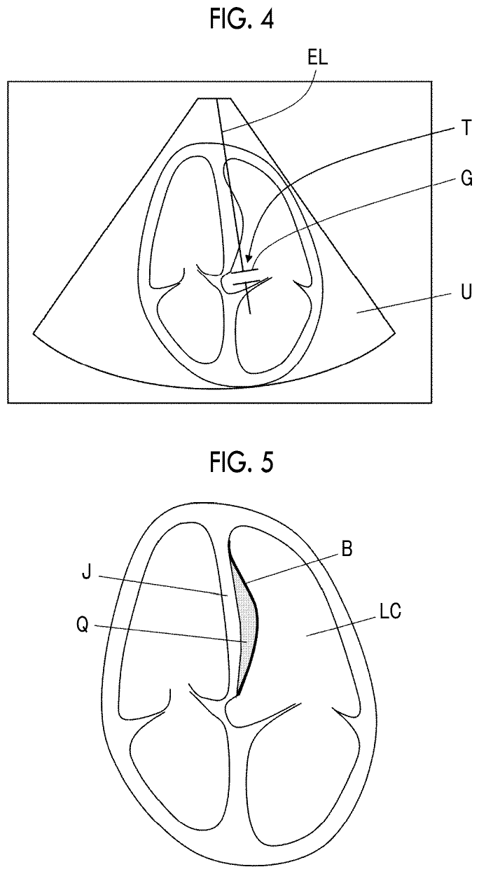

In the present invention, the image acquisition unit 33 acquires an ultrasound image U representing a so-called apical five-chamber cross section as schematically shown in FIG. 4. The apical five-chamber cross section is a cross section of a heart including five interior cavities of a left ventricle, a left ventricular outflow tract, a left atrium, a right ventricle, and a right atrium.

The image memory 24 is a memory that stores the ultrasound image U acquired by an image acquisition unit 33. The user can use the ultrasound image U stored in the image memory 24, for example, to check the ultrasound image U after the examination. Here, as the image memory 24, for example, recording media such as a flash memory, a hard disk drive (HDD), a solid state drive (SSD), a flexible disk (FD), a magneto-optical disk (MO disk), a magnetic tape (MT), a random access memory (RAM), a compact disc (CD), a digital versatile disc (DVD), a secure digital card (SD card), or a universal serial bus memory (USB memory) can be used.

The gate setting unit 25 sets a so-called Doppler gate used to calculate a velocity time integral value of a blood flow by a so-called pulse Doppler method on the ultrasound image U acquired by the image acquisition unit 33. For example, as shown in FIG. 4, the gate setting unit 25 sets a Doppler gate G on a left ventricular outflow tract T on the ultrasound image U. A scanning line EL passing through a center point of the Doppler gate G is associated with the Doppler gate G.

The gate setting unit 25 can set the Doppler gate G at a position designated by the user via the input device 32, for example. In addition, the gate setting unit 25 can recognize the left ventricular outflow tract T shown in the ultrasound image U by analyzing the ultrasound image U, and set the Doppler gate G on the recognized left ventricular outflow tract T, for example. In this case, the gate setting unit 25 can recognize the left ventricular outflow tract T by, for example, a method of so-called template matching in which a typical image pattern of the left ventricular outflow tract T in the apical five-chamber cross section is stored and the ultrasound image U is searched using the image pattern, or a method of using a so-called trained model in machine learning in which an image pattern of the left ventricular outflow tract T in the apical five-chamber cross section is learned.

The boundary recognition unit 26 recognizes a boundary B between a ventricular septum J and a left ventricular cavity LC in the ultrasound image U by analyzing the ultrasound image U, as schematically shown in FIG. 5. In general, in the ultrasound image U, a brightness difference occurs between the ventricular septum J and the left ventricular cavity LC. Therefore, the boundary recognition unit 26 can calculate, for example, a line having a maximum brightness difference in the ultrasound image U, and recognize the calculated line as the boundary B.

In addition, the boundary recognition unit 26 can input the ultrasound image U acquired by the image acquisition unit 33 to a trained model in machine learning that has been trained in advance using a large number of ultrasound images U representing the apical five-chamber cross section and the boundary B between the ventricular septum J and the left ventricular cavity LC shown in the ultrasound images U, and output the boundary B in the input ultrasound image U to the trained model. It should be noted that a line having a maximum brightness difference can be used as the boundary B in the large number of ultrasound images U used for the learning, and a line designated by a specialist such as a skilled doctor can also be used.

The boundary B recognized by the boundary recognition unit 26 in this way may bulge toward the left ventricular cavity LC due to the subject or due to calcification of a part of the ventricular septum J. FIG. 5 shows an example in which the boundary B bulges toward the left ventricular cavity LC due to a calcified portion Q of the ventricular septum J.

The overlap determination unit 27 determines whether or not there is an overlap between the scanning line EL passing through the center point of the Doppler gate G set by the gate setting unit 25 and the boundary B between the ventricular septum J and the left ventricular cavity LC recognized by the boundary recognition unit 26, for the plurality of frames of the ultrasound images U continuously acquired by the image acquisition unit 33.

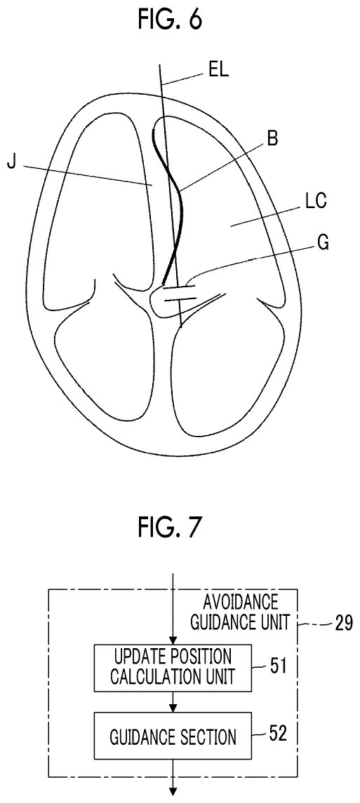

In general, since a structure in the heart changes in time series due to the pulsation of the heart, in a case where the Doppler gate G is set on the left ventricular outflow tract T, a positional relationship between a position of the ventricular septum J and a position of the scanning line EL associated with the Doppler gate G in the ultrasound image U may also change due to the pulsation. In particular, in a case where the ventricular septum J bulges toward the left ventricular cavity LC due to some factor such as calcification, a state in which the boundary B and the scanning line EL overlap each other and a state in which the boundary B and the scanning line EL are separated from each other may alternately appear in time series due to the pulsation, as schematically shown in FIG. 6.

The time-series change determination unit 28 determines whether or not the overlap determination result by the overlap determination unit 27, that is, the determination result of whether or not the boundary B between the ventricular septum J and the left ventricular cavity LC and the scanning line EL passing through the center point of the Doppler gate G overlap each other changes within the cardiac cycle. Since the position of the boundary B recognized by the boundary recognition unit 26 is periodically displaced due to the pulsation, the time-series change determination unit 28 can specify the cardiac cycle based on, for example, the time-series displacement of the boundary B.

Here, the measurement value obtained in the Doppler gate G, that is, for example, the measurement value obtained by the velocity time integral value calculation unit 30 as described below is affected by a structure overlapping the scanning line EL passing through the center point of the Doppler gate G. Therefore, in a case where the overlap determination result between the boundary B between the ventricular septum J and the left ventricular cavity LC and the scanning line EL changes within the cardiac cycle, the measurement value in the Doppler gate G is calculated while the structure in the heart overlapping the scanning line EL changes in time series. In this way, in a case where the measurement condition changes in time series within the cardiac cycle, the accurate velocity time integral value may not be measured in the Doppler gate G.

In a case where the time-series change determination unit 28 determines that the overlap determination result changes within the cardiac cycle, the avoidance guidance unit 29 guides the user to a method of avoiding the change in the overlap determination result.

The avoidance guidance unit 29 can guide the user to move the position of the Doppler gate G on the ultrasound image U, for example. In this case, as shown in FIG. 7, the avoidance guidance unit 29 can include an update position calculation unit 51 and a guidance section 52.

The update position calculation unit 51 calculates an update position of the Doppler gate G at which the scanning line EL passing through the center point of the Doppler gate G and the boundary B between the ventricular septum J and the left ventricular cavity LC do not overlap each other over the cardiac cycle. The update position calculation unit 51 can calculate, for example, a position closest to the center of the left ventricular outflow tract T among the positions of the Doppler gate G at which the scanning line EL and the boundary B do not overlap each other over the cardiac cycle, as the update position of the Doppler gate G.

The guidance section 52 guides the user to the update position calculated by the update position calculation unit 51. The user can, for example, accept the update position via the input device 32, and can further adjust the update position via the input device 32. In addition, the guidance section 52 can cause the gate setting unit 25 to set a new Doppler gate G based on the calculated update position.

In addition, the avoidance guidance unit 29 can also guide movement of the ultrasound probe 1 as a method of avoiding the change in the overlap determination result. The avoidance guidance unit 29 can guide, for example, the ultrasound probe 1 to slide in a right flank direction of the subject. In a case where the ultrasound probe 1 is slid in the right flank direction of the subject, the heart of the subject moves to the left in the ultrasound image U, so that the scanning line EL passing through the center point of the Doppler gate G moves relatively to the left ventricular cavity LC side with respect to the ventricular septum J. As a result, it is possible to avoid the overlap of the scanning line EL with the boundary B.

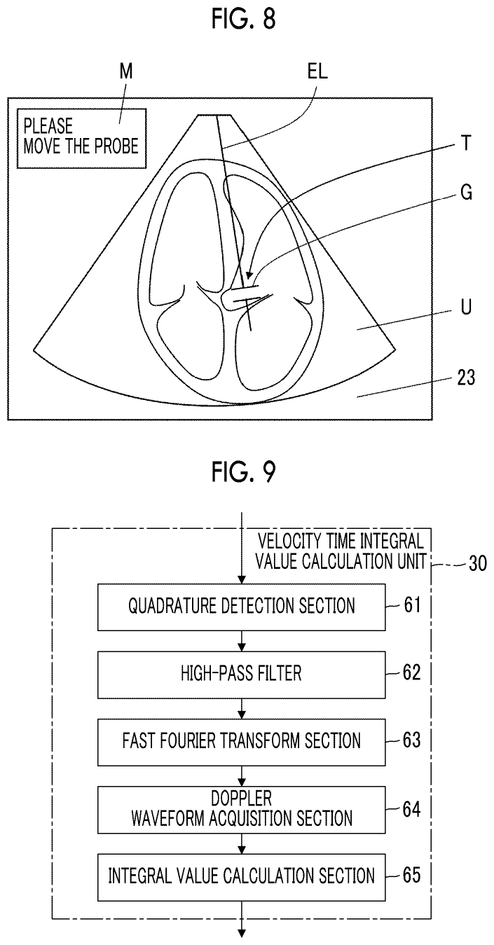

In a case where the movement of the ultrasound probe 1 is guided, the avoidance guidance unit 29 can display, for example, as shown in FIG. 8, a message M indicating the movement of the ultrasound probe 1 on the monitor 23. In a case where the ultrasound diagnostic apparatus comprises a speaker (not shown), the avoidance guidance unit 29 can also guide the user by a voice via the speaker.

The avoidance guidance unit 29 can also guide the measurement in an ultrasound image representing a so-called apical three-chamber cross section instead of the ultrasound image U representing the apical five-chamber cross section as a method of avoiding the change in the overlap determination result.

The velocity time integral value calculation unit 30 calculates the velocity time integral value of the blood flow in the Doppler gate G set by the gate setting unit 25. As shown in FIG. 9, the velocity time integral value calculation unit 30 has a configuration in which a quadrature detection section 61, a high-pass filter 62, a fast Fourier transform section 63, a Doppler waveform acquisition section 64, and an integral value calculation section 65 are connected in series.

The quadrature detection section 61 mixes the sound ray signal received from the transmission/reception circuit 12 with a carrier signal of a reference frequency to orthogonally detect the sound ray signal and convert the sound ray signal into a complex signal.

The high-pass filter 62 functions as a so-called wall filter, and removes a frequency component derived from the motion of the body tissue inside the subject, from the complex signal generated by the quadrature detection section 61.

The fast Fourier transform section 63 performs a Fourier transform on the complex signal of a plurality of sample points to perform frequency analysis, obtains the blood flow velocity, and generates a spectrum signal.

The Doppler waveform acquisition section 64 acquires a Doppler waveform image signal by aligning the spectrum signals generated by the fast Fourier transform section 63 on a time axis and expressing the magnitude of each frequency component in brightness. The Doppler waveform image signal is called a so-called Doppler image, in which the lateral axis indicates a time axis, the vertical axis indicates a Doppler shift frequency, that is, a flow velocity, and the brightness of the waveform represents power in each frequency component.

The integral value calculation section 65 specifies the cardiac cycle in the Doppler waveform image signal, and calculates the velocity time integral value by time-integrating the value of the flow velocity in the Doppler waveform image signal over the cardiac cycle. The integral value calculation section 65 can specify a repetition unit of the waveform in the Doppler waveform image signal by analyzing the Doppler waveform image signal, and can specify a period to which the specified repeating unit belongs as the cardiac cycle, for example.

Since the Doppler gate G is set on the left ventricular outflow tract T on the ultrasound image U by the gate setting unit 25, the velocity time integral value calculated by the velocity time integral value calculation unit 30 in this way is a value obtained by integrating the velocity of the blood flow flowing through the left ventricular outflow tract T over the cardiac cycle. The velocity time integral value can be used to calculate a so-called stroke volume and a cardiac output together with the diameter of the left ventricular outflow tract T measured in the ultrasound image representing the so-called parasternal left ventricular long-axis cross section or the like.

It should be noted that the processing of calculating the velocity time integral value by the quadrature detection section 61, the high-pass filter 62, the fast Fourier transform section 63, the Doppler waveform acquisition section 64, and the integral value calculation section 65 is performed after the transducer array 11 is driven by sending the drive signal under the control of the transmission/reception circuit 12 and the apparatus control unit 31 to transmit the ultrasound waves in a pulsed manner.

The display controller 22 performs predetermined processing on the ultrasound image U acquired by the image acquisition unit 33 and the guidance or the like by the avoidance guidance unit 29 under the control of the apparatus control unit 31, and displays the processed image on the monitor 23.

The monitor 23 displays the ultrasound image U and the guidance or the like by the avoidance guidance unit 29 under the control of the display controller 22, and includes, for example, a display device such as a liquid crystal display (LCD) or an organic electroluminescence display (organic EL display).

The input device 32 is an input device for the user to perform an input operation, and is configured by, for example, a device such as a keyboard, a mouse, a trackball, a touchpad, and a touch sensor disposed in a state of being superimposed on the monitor 23.

In the present embodiment, each processing in the processor 34 is executed by any computer. In addition, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a computer for specific use, a workstation, or another system capable of executing each processing.

The processor 34 may be composed of one or a plurality of pieces of hardware, and types of hardware are not limited. For example, the processor 34 may be composed of hardware such as a central processing unit (CPU), a micro processing unit (MPU), a programmable logic device such as a field programmable gate array (FPGA), a dedicated circuit for executing specific processing, such as an application specific integrated circuit (ASIC), a graphic processing unit (GPU), or a neural processing unit (NPU). Further, the type of hardware may be a combination of different types of hardware. In a case in which the plurality of types of hardware are configured to execute one or a plurality of types of processing of a certain processor, the plurality of types of hardware may exist in devices physically separated from each other or may exist in the same device. Furthermore, in any of the embodiments, the order of each processing performed by the processor 34 is not limited to the above-described order, and may be changed as appropriate. The hardware is composed of an electric circuit (circuitry) in which circuit elements such as semiconductor elements are combined.

The program may be software such as firmware or a microcode. Furthermore, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media existing in physically separated devices. The program code or the code segment may represent any combination of procedures, functions, subprograms, routines, subroutines, modules, software packages, classes, instructions, data structures, or program statements. The program code or the code segments may be connected to other code segments or hardware circuits by transmitting and receiving information, data, an argument, a parameter, or content of a memory.

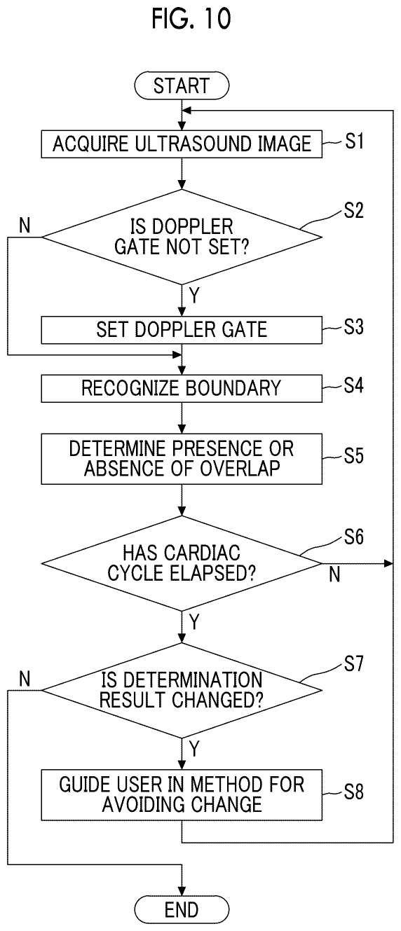

Next, an operation of the ultrasound diagnostic apparatus according to the embodiment will be described with reference to a flowchart shown in FIG. 10.

In step S1, the image acquisition unit 33 acquires the ultrasound image U representing the apical five-chamber cross section. In this case, under the control of the apparatus control unit 31, the transmission and reception of the ultrasound from the plurality of transducers of the transducer array 11 are started in accordance with the drive signal from the pulser 41 of the transmission/reception circuit 12 of the ultrasound probe 1, the ultrasound echo from the subject is received by the plurality of transducers of the transducer array 11, and the reception signal as the analog signal is output to the amplifying unit 42, is amplified, and then is subjected to the AD conversion via the AD conversion unit 43 to acquire the reception data.

The reception focus processing is performed on the reception data by the beam former 44, the sound ray signal generated by the reception focusing processing is transmitted to the image generation unit 21 of the apparatus main body 2, and thus the ultrasound image U is generated by the image generation unit 21. In this case, the signal processing unit 45 of the image generation unit 21 performs the correction of the attenuation in accordance with the depth of the reflection position of the ultrasound and the envelope detection processing on the sound ray signal, the DSC 46 performs the conversion into the image signal in accordance with the normal television signal scanning method, and the image processing unit 47 performs various types of necessary image processing such as gradation processing. The ultrasound image U generated in step S1 in this way is transmitted to the display controller 22, the image memory 24, the gate setting unit 25, and the boundary recognition unit 26.

In step S2, the gate setting unit 25 determines whether or not the Doppler gate G is not set for the ultrasound image U acquired in step S1 by referring to a setting history of the Doppler gate G or the like. In a case where it is determined in step S2 that the Doppler gate G is not set, the processing proceeds to step S3. In addition, in a case where it is determined in step S2 that the Doppler gate G is set, step S3 is skipped and the processing proceeds to step S4. Since the Doppler gate G is not set at the current point in time, the processing proceeds to step S3.

In step S3, as shown in FIG. 4, the gate setting unit 25 sets the Doppler gate G used to calculate the velocity time integral value of the blood flow by the pulse Doppler method on the ultrasound image U acquired in step S1. The gate setting unit 25 can set the Doppler gate G at a position designated by the user via the input device 32, for example. In addition, the gate setting unit 25 can recognize the left ventricular outflow tract T shown in the ultrasound image U by analyzing the ultrasound image U, and set the Doppler gate G on the recognized left ventricular outflow tract T, for example.

In step S4, as schematically shown in FIG. 5, the boundary recognition unit 26 recognizes the boundary B between the ventricular septum J and the left ventricular cavity LC in the ultrasound image U by analyzing the ultrasound image U acquired in step S1. The boundary recognition unit 26 can calculate, for example, a line having a maximum brightness difference in the ultrasound image U, and recognize the calculated line as the boundary B. In addition, the boundary recognition unit 26 can recognize the boundary B by using a trained model in machine learning that has been trained in advance using a large number of ultrasound images U representing the apical five-chamber cross section and the boundary B between the ventricular septum J and the left ventricular cavity LC shown in the ultrasound images U.

In step S5, the overlap determination unit 27 determines whether or not there is an overlap between the scanning line EL passing through the center point of the Doppler gate G set by the gate setting unit 25 and the boundary B between the ventricular septum J and the left ventricular cavity LC recognized by the boundary recognition unit 26, for the ultrasound image U acquired in step S1. The determination result is stored in the time-series change determination unit 28.

In step S6, the time-series change determination unit 28 determines whether or not the cardiac cycle has elapsed, that is, whether or not the plurality of frames of the ultrasound images U have been acquired over the cardiac cycle. Since the boundary B between the ventricular septum J and the left ventricular cavity LC recognized in step S4 is periodically displaced due to the pulsation, the time-series change determination unit 28 can specify the cardiac cycle based on, for example, the time-series displacement of the boundary B.

In a case where it is determined in step S6 that the cardiac cycle has not elapsed, the processing returns to step S1, and the ultrasound image U is newly acquired. In subsequent step S2, it is determined whether or not the Doppler gate G is not set, but since the Doppler gate G is already set at the current point in time, it is determined that the Doppler gate G is set, and the processing proceeds to step S4. In step S4, the boundary B between the ventricular septum J and the left ventricular cavity LC is recognized, and in step S5, it is determined whether or not the scanning line EL passing through the center point of the Doppler gate G and the boundary B between the ventricular septum J and the left ventricular cavity LC overlap each other.

In this way, the processing of steps S1 to S6 is repeated as long as it is determined in step S6 that the cardiac cycle has not elapsed. As a result, a plurality of overlap determination results of step S5 for the plurality of frames of the ultrasound images U that are continuous in time are accumulated in the time-series change determination unit 28. In a case where it is determined in step S6 that the cardiac cycle has elapsed, the processing proceeds to step S7.

In step S7, the time-series change determination unit 28 determines whether or not the overlap determination result obtained in step S5 changes within the cardiac cycle. In a case where it is determined in step S7 that the overlap determination result changes within the cardiac cycle, the processing proceeds to step S8.

The measurement value obtained in the Doppler gate G, that is, for example, the measurement value obtained by the velocity time integral value calculation unit 30 is affected by a structure overlapping the scanning line EL passing through the center point of the Doppler gate G. Therefore, in a case where the overlap determination result between the boundary B between the ventricular septum J and the left ventricular cavity LC and the scanning line EL changes within the cardiac cycle, the measurement value in the Doppler gate G is calculated while the structure in the heart overlapping the scanning line EL changes in time series. In this way, in a case where the measurement condition changes in time series within the cardiac cycle, the accurate velocity time integral value may not be measured in the Doppler gate G.

Therefore, in step S8, the avoidance guidance unit 29 guides the user in a method for avoiding the change in the overlap determination result. The avoidance guidance unit 29 can guide the user to move the position of the Doppler gate G on the ultrasound image U, for example.

In addition, the avoidance guidance unit 29 can calculate the update position of the Doppler gate G at which the scanning line EL passing through the center point of the Doppler gate G and the boundary B between the ventricular septum J and the left ventricular cavity LC do not overlap each other over the cardiac cycle, and guide the user to the calculated update position.

In addition, the avoidance guidance unit 29 can also guide movement of the ultrasound probe 1 as a method of avoiding the change in the overlap determination result. In this case, the avoidance guidance unit 29 can guide, for example, the ultrasound probe 1 to slide in the right flank direction of the subject.

In addition, the avoidance guidance unit 29 can also guide the measurement in an ultrasound image representing a so-called apical three-chamber cross section instead of the ultrasound image U representing the apical five-chamber cross section as a method of avoiding the change in the overlap determination result.

In a case where step S8 is completed, the processing of steps S1 to S8 is performed again. In this way, the processing of steps S1 to S8 is repeated as long as it is determined in step S7 that the overlap determination result within the cardiac cycle changes. In a case where it is determined in step S7 that the overlap determination result does not change within the cardiac cycle, step S8 is skipped, and the operation of the ultrasound diagnostic apparatus according to the flowchart of FIG. 10 is completed.

The user can set the Doppler gate G at a position on the ultrasound image U at which the scanning line EL passing through the center point of the Doppler gate G and the boundary B between the ventricular septum J and the left ventricular cavity LC do not overlap each other over the cardiac cycle by adjusting the position of the Doppler gate G on the ultrasound image U, changing the position of the ultrasound probe 1, imaging the ultrasound image representing the apical three-chamber cross section, and the like in accordance with the method guided in step S8.

After the Doppler gate G is set in this way, the velocity time integral value of the blood flow is calculated in the Doppler gate G by the velocity time integral value calculation unit 30 using the pulse Doppler method. Since the scanning line EL passing through the center point of the Doppler gate G and the boundary B between the ventricular septum J and the left ventricular cavity LC do not overlap each other over the cardiac cycle, the velocity time integral value can be accurately calculated.

As described above, according to the ultrasound diagnostic apparatus of the embodiment of the present invention, the boundary recognition unit 26 recognizes the boundary B between the ventricular septum J and the left ventricular cavity LC in the ultrasound image U, the overlap determination unit 27 determines whether or not there is an overlap between the scanning line EL passing through the center point of the Doppler gate G and the recognized boundary B, the time-series change determination unit 28 determines whether or not the overlap determination result changes within the cardiac cycle, and the avoidance guidance unit 29 guides the user in a method for avoiding the change in the overlap determination result in a case where it is determined that the overlap determination result changes within the cardiac cycle, so that the Doppler gate G can be disposed at an appropriate position to obtain an accurate Doppler measurement value.

A case has been described in which the transmission/reception circuit 12 is provided in the ultrasound probe 1, but the transmission/reception circuit 12 may be provided in the apparatus main body 2.

Further, a case has been described in which the image generation unit 21 is provided in the apparatus main body 2, but the image generation unit 21 may be provided in the ultrasound probe 1.

The apparatus main body 2 may be a so-called stationary type, a portable type that is easily carried, or a so-called handheld type that is configured by, for example, a smartphone or a tablet type computer. In this way, the type of the device constituting the apparatus main body 2 is not particularly limited.

It has been described that the boundary recognition unit 26 recognizes the boundary B between the ventricular septum J and the left ventricular cavity LC in each of the plurality of frames of the ultrasound image U acquired by the image acquisition unit 33, but the processing of tracking the boundary B by using the information on the boundary B recognized in the first frame of the ultrasound image U can also be performed on the second frame or subsequent frames of the ultrasound image U. In this case, the boundary recognition unit 26 can use, for example, a known algorithm such as so-called template matching or so-called optical flow using the boundary B recognized in the first frame of the ultrasound image U as template data.

In addition, in accordance with the guidance of the avoidance guidance unit 29, as a result of the user adjusting the position of the Doppler gate G on the ultrasound image U, changing the position of the ultrasound probe 1, imaging the ultrasound image representing the apical three-chamber cross section, and the like, the position of the Doppler gate G in the ultrasound image U is adjusted. As a result, the measurement direction of the blood flow by the Doppler gate G, that is, the direction orthogonal to the scanning line EL passing through the center point of the Doppler gate G may deviate from the direction of the blood flow to be measured, that is, the traveling direction of the left ventricular outflow tract T. The value of the component of the original blood flow velocity along the measurement direction is obtained by the Doppler gate G, but in a case where the direction orthogonal to the scanning line EL deviates from the traveling direction of the left ventricular outflow tract T, a difference between the measured value of the blood flow velocity and the original value increases. Therefore, the velocity time integral value calculation unit 30 can specify the traveling direction of the left ventricular outflow tract T by analyzing the ultrasound image U or the like, set the blood flow perpendicular line orthogonal to the specified traveling direction of the left ventricular outflow tract T, and correct the velocity time integral value based on the angle between the set blood flow perpendicular line and the scanning line EL.

The velocity time integral value calculation unit 30 can specify the traveling direction of the left ventricular outflow tract T based on, for example, the brightness of the inner wall of the left ventricular outflow tract T in the ultrasound image U. The velocity time integral value calculation unit 30 can also specify the traveling direction of the left ventricular outflow tract T by using a trained model in machine learning that has been trained using the left ventricular outflow tract T and the traveling direction in the ultrasound image U. In a case where an angle between the blood flow perpendicular line orthogonal to the traveling direction of the left ventricular outflow tract T and the scanning line EL passing through the center point of the Doppler gate G is A, the velocity time integral value calculated based on the Doppler gate G is MV1, and the corrected velocity time integral value is MV2, a simple relationship of MV2=MV1×cos(A) is established, so that the corrected velocity time integral value can be calculated by MV1=MV2/cos(A). Here, cos is a cosine function.

### EXPLANATION OF REFERENCES

1: ultrasound probe
2: apparatus main body
11: transducer array
12: transmission/reception circuit
21: image generation unit
22: display controller
23: monitor
24: image memory
25: gate setting unit
26: boundary recognition unit
27: overlap determination unit
28: time-series change determination unit
29: avoidance guidance unit
30: velocity time integral value calculation unit
31: apparatus control unit
32: input device
33: image acquisition unit
34: processor
41: pulser
42: amplifying unit
43: AD conversion unit
44: beam former
45: signal processing unit
46: DSC
47: image processing unit
51: update position calculation unit
52: guidance section
61: quadrature detection section
62: high-pass filter
63: fast Fourier transform section
64: Doppler waveform acquisition section
65: integral value calculation section
B: boundary
EL: scanning line
G: Doppler gate
J: ventricular septum
M: message
LC: left ventricular cavity
Q: calcified portion
T: left ventricular outflow tract
U: ultrasound image

What is claimed is:

1. An ultrasound diagnostic apparatus comprising:
an ultrasound probe; and
a processor configured to:
acquire an ultrasound image in which a heart of a subject is imaged by transmitting and receiving an ultrasound beam using the ultrasound probe;
set a Doppler gate on the ultrasound image;
recognize a boundary between a ventricular septum and a left ventricular cavity in the ultrasound image;
determine whether or not an overlap between a scanning line passing through a center point of the Doppler gate and the boundary is present;
determine whether or not an overlap determination result of the scanning line and the boundary changes within one cardiac cycle; and
upon determining that the overlap determination result changes within the one cardiac cycle, guide a user in a method for avoiding a change in the overlap determination result.

2. The ultrasound diagnostic apparatus according to claim 1,
wherein the processor is configured to guide movement of a position of the Doppler gate on the ultrasound image.

3. The ultrasound diagnostic apparatus according to claim 2,
wherein the processor is configured to calculate an update position of the Doppler gate at which the scanning line and the boundary do not overlap each other over the one cardiac cycle.

4. The ultrasound diagnostic apparatus according to claim 3,
wherein the processor is configured to guide the user to the update position.

5. The ultrasound diagnostic apparatus according to claim 3,
wherein the processor is configured to set a new Doppler gate based on the update position.

6. The ultrasound diagnostic apparatus according to claim 1,
wherein the processor is configured to guide movement of the ultrasound probe.

7. The ultrasound diagnostic apparatus according to claim 6,
wherein the processor is configured to guide the ultrasound probe to slide in a right flank direction of the subject.

8. The ultrasound diagnostic apparatus according to claim 1,
wherein the processor is configured to:
acquire the ultrasound image in which an apical five-chamber cross section is imaged; and
guide measurement in an apical three-chamber cross section.

9. A control method of an ultrasound diagnostic apparatus comprising:
acquiring an ultrasound image in which a heart of a subject is imaged by transmitting and receiving an ultrasound beam using an ultrasound probe;
setting a Doppler gate on the ultrasound image;
recognizing a boundary between a ventricular septum and a left ventricular cavity in the ultrasound image;
determining whether or not an overlap between a scanning line passing through a center point of the set Doppler gate and the recognized boundary is present;
determining whether a determination result regarding the presence or absence of overlap changes within one cardiac cycle; and
guiding a user in a method for avoiding a change in the determination result regarding the presence or absence of overlap in a case where it is determined that the determination result regarding the presence or absence of overlap changes within the one cardiac cycle.
